(19) United States
(12) Patent Application Publication
(10) Pub. No.: US 2023/0225974 A1
(43) Pub. Date: Jul. 20, 2023

(54) SOLID SOLUTION MADE FROM GUM ARABIC AND AT LEAST ONE LIPOSOLUBLE ACTIVE SUBSTANCE

(71) Applicant: Metabolic Tuning AG, Tuebach (CH)

(72) Inventor: Hanspeter Strobel, Davos Platz (CH)

(73) Assignee: Metabolic Tuning AG, Tuebach (CH)

(21) Appl. No.: 17/998,188

(22) PCT Filed: May 11, 2021
(86) PCT No.: PCT/EP2021/062476

§ 371 Date: Nov. 8, 2022

(30) Foreign Application Priority Data
May 12, 2020 (EP) 20174201.2

(51) Int. Cl. A61K 9/14; A61K 31/122; A61K 9/00; A23L 29/25; A23L 2/52; A23L 2/395

(52) U.S. Cl. CPC A61K 9/148; A23L 2/52; A23L 2/395; A23L 29/25; A61K 9/0095; A61K 31/122

(57) ABSTRACT

Solid solution of gum arabic and at least one liposoluble active ingredient having particles of a size of less than 100 µm and a weight ratio of gum arabic to liposoluble active ingredients of 5:1 to 200:1, obtainable by drying an aqueous solution of gum arabic to achieve a fine-particle dry product; admixing the dry product with a solution or dispersion of the liposoluble active ingredient(s) in an anhydrous solvent in which gum arabic is insoluble; mixing in the solution while homogenizing and further comminuting the dry product to a particle size of less than 100 µm; and removing the solvent at a temperature of less than 70° C., as well as method for the preparation of the solution and the aqueous suspension containing it.

The invention relates to a solid solution of gum arabic and at least one liposoluble active substance comprising particles of a size of less than 100 µm and a weight ratio of gum arabic to liposoluble active substance of between 5:1 and 200:1, and to a method for preparing such solid solutions and the aqueous suspensions containing them.

Administering liposoluble active ingredients orally often causes a problem due to the fact that the active substances are not absorbed by the body from an aqueous environment which prevails in the intestine and on mucous membranes. Even when administered via a lipophilic carrier, the uptake rate often falls short of expectations.

For that reason, it was proposed to administer the active ingredients bound in micelles to improve their absorption in the body. Micelles are formed of substances that have a lipophilic end and a hydrophilic end and are thus capable of entrapping and transporting lipophilic substances in an aqueous environment. Examples in this context include a variety of detergents, polysorbates, poloxamers as well as a great number of natural substances, such as various natural resins and also gum arabic.

Using poloxamers for the solubilization of active ingredients, including insulin has been described in publication WO 2007/104173 A2. Reference is also made to the basic suitability of resins for these purposes.

In WO 2008/034273 A1 compositions based on gum arabic have been described, which is present in the form of micelles with an active ingredient bound therein, the compositions being obtained from a warm aqueous solution. The composition is liquid and contains micelles having a diameter ranging between 2 and 300 nm.

What all these approaches have in common is that they do not fundamentally improve the absorption of the respective active substance in the body.

The objective of the present invention is to provide an agent by means of which the absorption of liposoluble active substances in the body can be made more efficient. At the same time, a method for the preparation of this agent shall be presented and examples shall be given relating to the administration of this agent.

The size of the particles formed from gum arabic and the active ingredients has an influence on the administration mode as well as on the distribution of the active ingredient(s). On the one hand, the particle size is governed and determined by the production method and as well by the ratio of gum arabic to active ingredient.

Gum arabic exhibits a suspending and emulsion-stabilizing effect. It forms a protective colloid coating around liposoluble substances, which can be used to form a solid solution consisting of gum arabic and liposoluble active ingredients, and which facilitates their transport and absorption by an organism.

Accordingly, the invention relates to a solid solution consisting of gum arabic and at least one liposoluble active ingredient comprising particles of a size of less than 100 µm and a weight ratio of gum arabic to liposoluble active ingredients of between 20:1 and 200:1, obtainable by drying an aqueous solution of gum arabic to achieve a fine-particle dry product; admixing the dry product with a solution of the liposoluble active ingredient(s) in an anhydrous solvent in which gum arabic is not soluble; mixing in the solution while homogenizing and further fragmenting the dry product to a particle size of less than 100 µm; and removing the solvent at a temperature below the decomposition temperatures of gum arabic, in particular at less than 70° C.

Moreover, the invention also relates to a method for the preparation of a solid solution consisting of gum arabic and at least one liposoluble active ingredient, characterized by the steps of drying an aqueous solution of gum arabic to achieve a fine-particle dry product; admixing the fine-particle dry product with an anhydrous solution or dispersion of the liposoluble active ingredient or ingredients in a solvent in which gum arabic is insoluble, with a weight ratio of gum arabic to liposoluble active ingredients of between 20:1 and 200:1; admixing the solution while homogenizing and further fragmenting the dry product to a particle size of less than 100 µm; and removing the solvent at a temperature ≤ 70° C., preferably ≤ 40° C., in particular at room temperature.

It is essential for the understanding that the particle sizes mentioned define the maximum threshold. Accordingly, “particles of a size of less than 100 µm” means that the particles are far predominantly smaller than 100 µm. It is not to be viewed to denote a mean particle size of a statistical distribution. This applies to both the lower and upper thresholds.

Moreover, it is also an essential requirement that the production method is carried out at a temperature ≤ 70° C., preferably ≤ 40° C., and in particular at room temperature. The required fineness cannot be achieved from the solution or melt. Gum arabic can dissolve in the presence of a little amount of water at a temperature of between 40° C. and 60° C. The solubility of gum arabic in water is about 500 g/l.

As proposed by the present invention, the solid solution consisting of gum arabic and the active ingredient(s) is present in aqueous medium in the form of micelles. Micelles as a transport medium for fatty active ingredients have been used for decades to solubilize substances that are insoluble in water. After all, the cleaning effect of surfactants is based on the formation of micelles.

Micelles are aggregates of surfactant molecules the interior of which in an aqueous environment consists of lipophilic groups of the individual surfactant molecules, with the hydrophilic groups being located on the surface. These hydrophilic groups are capable of water accumulation and lead to the formation of an emulsion.

In their simplest form, micelles are of spherical shape to which they spontaneously self-organize. The remaining residues inside are not in contact with the surrounding aqueous phase. The diameter of the micelles is defined by the space requirement of the lipophilic core and the surrounding hydrophilic shell. They can be thought of as drops of liquid in their respective environments.

In the event there is an oversupply of surfactant molecules, the diameters of the aggregates increase significantly to values well exceeding ten times the smallest possible diameter. For that reason, it is essential to keep the ratio of gum arabic to the active substance(s) within the range claimed in invention, as this limits the particle size of the micelles formed.

In a micelle, the individual surfactant molecules in an aqueous environment can escape at any time from the micelle into the surrounding phase and, conversely, free surfactant molecules can aggregate with active ingredients to form new micelles, so that a constant exchange takes place. This equilibrium between the micelle and free surfactant and active ingredient molecules enables via a permeable membrane the transfer of the active ingredient into and through the membrane.

The transport effect of the inventive particles of gum arabic and liposoluble active ingredient is therefore based on the fact that the active ingredient is transported in an aqueous environment from the micelle to the transfer site where it is released. Upon release, the active ingredient enters the cells through the membrane.

Gum arabic is obtained from the sap of various acacias (for example Acacia senegal, Acacia seyal) and primarily consists of the acid alkali and alkaline earth salts of arabic acid (polyarabinic acid). Polyarabinic acid is a branched polysaccharide consisting of L-arabinose, D-galactose, L-rhamnose and D-glucuronic acid in the ratio of 3:3:1:1 and having a molecular weight in the range of 200,000 to 350,000 g/mol, depending on the measurement method applied.

It is used as a thickener, emulsifier and stabilizer in food industrial applications. It is further employed in the beverage industry, confectionery, pharmaceuticals, cosmetics and engineering sectors.

The invention basically provides for the use of all gum arabic grades. Prerequisite in this context is that a certain degree of fine-particle size already exists prior to the inventive processing, which is achieved in particular in the case of spray drying from an aqueous solution. However, the particle size after spray drying is not yet suited for the purposes proposed by the invention, because it is still too coarse. Gum arabic powder of good quality obtained by spray drying is commercially available with a bulk density of 600 kg/m³ and an internal specific surface area of ≥ 150 m²/g. This capillarity of the waterproof powder is available for the entrapment of the lipophilic active ingredient(s). Further comminution then results in the formation of the particles proposed by the invention.

Therefore, the gum arabic powder to be employed should be of amorphous structure with many capillaries and a large internal surface area. The large internal surface area is an important requirement for the active ingredient solution to be “soaked up” or properly dispersed and distributed as evenly as possible. At the same time, good water solubility is essential, but this is regularly ensured by a product obtained by spray drying. This enables the production of highly concentrated solutions or suspensions of relatively low viscosity.

As proposed by the invention, the preparation method is implemented in the absence of water; the inventive solid solution obtained is anhydrous. This is essential for ensuring the stability of the product as well as for the usefulness of aqueous suspensions and solutions obtained from it.

The method proposed by the invention, as well as the ‘solid solution’ obtained as a result, is suitable for stabilizing these micelles for years. In this process, even if the active ingredient is in liquid form, the particles formed (micelles) are solid and obtained as a dry powder that, as a “semi-finished product”, can be easily incorporated into any galenical form (cream, serum, lotion, gel, drink, shot). The powder can be suspended without difficulty in small quantities of water or an aqueous solution -complete emulsion occurs only when the solubility limit is fallen below.

Using different active ingredient modules enables a variety of effects to be generated simply, economically and without major development effort. The effects of such active ingredient complexes are more rapidly achieved than with customary “carrier systems”.

According to the invention, gum arabic and active ingredient are used in a weight ratio of between 5:1 and 200:1, in particular in a weight ratio of 10:1 to 100:1. The weight ratio is an important factor because too high a proportion of gum arabic can lead to an increase of the size of the particles, which is detrimental to effective distribution.

As already indicated, the particle size is of essential significance. The particles of the solid solution should all be substantially sized ≤ 100 µm, preferably 80% of the particles shall have a size of ≤ 60 µm. Likewise, to achieve a homogeneous product, the minimum particle size should be limited as well, such that in particular 80% of the particles have a size ≥ 1 µm and in particular 90% of the particles have a particle size of ≥ 1 µm. Thus, the mean particle size is likely to be around 40 µm.

As far as the incorporation of the active ingredient(s) in solution is concerned, the fine-particle size and inner surface of the gum arabic matrix material used is crucial. The solution must be completely and as uniformly as possible absorbed and distributed within the matrix material. It is to be ensure in this respect that the solvent for the active ingredient(s) does not dissolve the gum arabic and thus does not change its structure.

Suitable solvents dissolve the respective lipophilic active ingredient and form solutions that are as highly concentrated as possible. The solvent should be anhydrous, and another criterion is that its boiling point is as low as possible which would enable the solvent to be eliminated from the product. Examples of suitable solvents include ethers and alcohols, in particular ethanol. Especially preferred is absolute alcohol (100%). The presence of water in the matrix material mixed with active ingredient must be avoided.

Major prerequisite for the preparation of a solid solution proposed by the present invention is that homogenization and further fragmentation of the matrix material impregnated with the active ingredient(s) takes place. Basically, any method that ensures the appropriate particle sizes are obtained can be used for this purpose; nevertheless, particularly preferred for these purposes is using cutters and homogenizers exerting high shear forces.

Homogenization or a final step of the production process causes the solvent of the active ingredient(s) to be removed. As regards highly volatile solvents, such as alcohol, a great amount of the solvent will volatilize already during homogenization and further fragmentation, with the remaining residue being allowed to evaporate at a temperature ≤ 70° C., preferably ≤ 40° C., and in particular at room temperature, or also being removed in vacuo.

Following this procedure results in the formation of a stable powder consisting of gum arabic and the active ingredient(s). This powder is readily suspendible in small quantities of water and soluble in larger volumes of water. The aqueous suspensions or solutions are also stable with no agglomeration occurring.

Encapsulated in gum arabic, the inventive solid solution preferably contains only one liposoluble active ingredient. It should nonetheless be understood that the invention also encompasses mixtures which can be obtained, on the one hand, via a production process simultaneously incorporating several liposoluble active ingredients into the gum arabic particles or solid solutions of several active ingredients mixed with one another. The same applies analogously to the production method.

The solid solutions proposed by the invention can be marketed in the form of powders, however they serve primarily as starting substances to prepare suspensions containing 2.5 to 40.0 wt% of the solid solution suspended in water. Suspensions of this nature may additionally contain customary food-grade additives, such as, for example, preservative agents, stabilizers, pH regulators, sweeteners, flavorings.

Moreover, water-soluble vitamins, provitamins, dietary supplements and nutrients may be added in usual amounts. Usual amounts in this context range from 30 to 300 % of a recommended daily allowance. The total content of these additives usually amounts to between 0.5 and 2.5 wt% of the aqueous suspension.

It is to be understood that in aqueous suspensions of this kind, part of the added particles consisting of gum arabic and liposoluble active ingredient goes into solution, depending on concentration. In a five percent aqueous suspension, a portion of gum arabic with encapsulated active ingredient is as a rule present in particulate form; a portion in the form of a film or sponge.

As provided by the inventive method, all active ingredients suitable for incorporation in dissolved form into the fine-particle gum arabic can basically be employed as liposoluble active ingredients. Examples include coenzyme Q10, coenzyme Q10H2, vitamin A, vitamin D, vitamin E (tocopherol), vitamin K, fat-soluble vitamins of the B group, curcumin, chalcones, flavan-3-ols, flavanones, Flavones, flavonols, isoflavones, flavanolols, essential fatty acids, growth factors, immunoglobulins, immunoregulators, hormones (progesterone, testosterone, estrone, estradiol, androstenedione, etc.) as well as numerous fat-soluble drugs (analgesics, anti-inflammatory agents, antibiotic agents).

### EXAMPLE

Coenzyme Q10 (ubiquinone) was used as active ingredient. The procedure involved 100 g of gum arabic powder obtained by spray drying to be mixed with 2.0 g of ubiquinone suspended in 10 ml of absolute alcohol and homogenized in a cutter (Robocoupe) for 90 sec. and fragmented to achieve a particle size of ≤ 100 µm. The obtained fine-particle powder of the solid solution of ubiquinone in gum arabic was allowed to stand at room temperature under light protection until the alcohol had evaporated. The powder is stable when stored in a cool place protected from light.

51 g of the powder thus obtained were added to 946.5 g of distilled water together with 0.5 g of citric acid (pH regulator), 1 g of ascorbic acid (stabilizer) and 1 g of potassium sorbate (preservative agent), resulting in a suspension that remained stable for months. This suspension is ready to drink and suitable for immediate oral ingestion of ubiquinone.

Other substances may be added to the suspension obtained in this way, such as water-soluble vitamins, provitamins, dietary supplements, nutrients, sweeteners and flavors. The addition of vitamins, turmeric extract (curcumin) and passion fruit extract (chrysin) has proven to be particularly suitable. These and other additives can provide escorting assistance.

Sodium hydrogen carbonate (about 4 g/l) may be added as a release agent. It has been found that the additive accelerates the dissolution of the powder, and in combination with vitamin C also causes foaming.

1. Solid solution of gum arabic and at least one liposoluble active ingredient having particles of a size of less than 100 µm and a weight ratio of gum arabic to liposoluble active ingredients of between 5:1 and 200:1, obtainable by drying of an aqueous solution of gum arabic to produce a fine-particle dry product; admixing the dry product with a solution or dispersion of the liposoluble active ingredient(s) in an anhydrous solvent in which gum arabic is insoluble; mixing in the solution while homogenizing and further fragmenting the dry product to a particle size of less than 100 µm; and removing the solvent at a temperature ≤ 70° C., preferably at ≤ 40° C.

2. Solid solution according to claim 1, characterized in that the particles consisting of gum arabic and at least one liposoluble active ingredient form micelles.

3. Solid solution according to claim 2, characterized in that the weight ratio of gum arabic to liposoluble active ingredients ranges between 50:1 and 100:1.

4. Solid solution according to claim 1, characterized in that 80% of the particles have a size of ≤ 60 µm.

5. Solid solution according to claim 4, characterized in that 80% of the particles have a size of ≤ 40 µm.

6. Solid solution according to claim 1, characterized in that the fine-particle dry product is spray-dried gum arabic.

7. Solid solution according to claim 1, characterized in that the solvent of the solution or dispersion of the liposoluble active ingredient(s) is absolute alcohol.

8. Method for preparing a solid solution of gum arabic and at least one liposoluble active ingredient, characterized by the steps of drying of an aqueous solution of gum arabic to produce a fine-particle dry product; adding to the fine-particle dry product a solution or dispersion of the liposoluble active ingredient(s) in an anhydrous solvent in which gum arabic is insoluble, in a weight ratio of gum arabic to liposoluble active ingredient(s) ranging between 20:1 and 200:1; mixing in the solution while homogenizing and further fragmenting the dry product to a particle size smaller than 100 µm; and removing the solvent at a temperature of ≤ 70° C., preferably at ≤ 40° C.

9. Method according to claim 8, characterized in that spray-dried gum arabic is used as dry product.

10. Method according to claim 8, characterized in that further fragmentation is performed by means of a cutter or sonicator.

11. Method according to claim 8, characterized in that further fragmentation is carried out under high shear until 80% of the particles have a size of ≤ 60 µm.

12. Method according to claim 11, characterized in that further fragmentation is performed until 80% of the particles have a size of ≤ 40 µm.

13. Method according to claim 8, characterized in that a weight ratio of gum arabic to liposoluble active ingredients of 1:50 to 1:100 is used.

14. Method according to claim 8, characterized in that absolute alcohol is used as solvent.

15. Aqueous suspension containing 2.0 to 40.0 wt% of the solid solution of gum arabic according to claim 1.

16. Aqueous suspension according to claim 15, characterized in that said suspension additionally contains customary food-grade preservative agents, stabilizers, pH regulators, sweeteners and/or flavoring agents in an amount, taken together, of 0.05 to 1.0 wt%.

17. Aqueous suspension according to claim 15, characterized in that said suspension additionally contains water-soluble vitamins, provitamins, nutrients and/or nutritional supplements in an amount, taken together, of 0.5 to 2.5 wt%.

18. Solid solution according to claim 1, characterized in that the active ingredient or one of the active ingredients is co-enzyme Q10 and/or curcumin.
